(19) United States
(12) Patent Application Publication
(10) Pub. No.: US 2011/0004099 A1
(43) Pub. Date: Jan. 6, 2011

(54) METHOD AND APPARATUS USING ULTRASOUND FOR ASSESSING INTRACARDIAC PRESSURE

(75) Inventor: Antony Y. Kim, Portland, OR (US)

Correspondence Address: Schwabe Williamson & Wyatt;PACWEST CENTER, SUITE 1900, 1211 SW FIFTH AVENUE, PORTLAND, OR, 97204, US

(73) Assignee: Oregon Health & Science University, Portland, OR (US)

(21) Appl. No.: 12/921,320

(22) PCT Filed: Mar. 9, 2009
(86) PCT No.: PCT/US09/36551

§ 371 Date: Sep. 7, 2010

Related U.S. Application Data
(60) Provisional application No. 61/034,909, filed on Mar. 7, 2008.

(51) Int. Cl. A61B 8/00

(52) U.S. Cl. 600/438

(57) ABSTRACT

Embodiments relate to the field of hemodynamics, and, more specifically, to non-invasive methods of intracardiac pressure assessment. Some embodiments include acquiring ultrasound image data of a right internal jugular (IJ) vein in a subject, processing the ultrasound image data to determine vascular characteristic data for the IJ vein, and determining the right-sided intracardiac pressure from the vascular characteristic data. Also disclosed are systems and apparatus for carrying out the methods.

### CROSS REFERENCE TO RELATED APPLICATIONS

The present application claims priority to U.S. Provisional Patent Application No. 61/034,909, filed Mar. 7, 2008, entitled “ULTRASOUND BASED APPROACH TO DIAGNOSING HEART FAILURE,” the disclosure of which is hereby incorporated by reference in its entirety.

### TECHNICAL FIELD

Embodiments herein relate to the field of hemodynamics, and, more specifically, to non-invasive methods and apparatus for intra-cardiac pressure assessment.

### BACKGROUND

The current standard for right-sided intracardiac pressure measurement is right-heart catheterization (RHC). Assessment of the right-sided intracardiac pressures is vital in estimating right heart function and volume status in patients with acute and chronic heart failure (CHF). It is also important in broad clinical settings, such as in volume management of chronic dialysis patients and critically-ill patients in the intensive care unit. However, the RHC procedure is invasive, and it is impractical to perform serial RHC. Thus, RHC is not a suitable technique for providing immediate up-to-date clinical information necessary for management of patients on a day-to-day basis.

An alternative, non-invasive procedure, the estimation of jugular venous pulsation (JVP) also has many limitations and is unreliable. Thus, it is not a suitable substitute for RHC.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIGS. 1A and 1B illustrate the correlation between the standard method of jugular venous pulsation (JVP) estimation and the intracardiac hemodynamics right atrial pressure in millimeters of mercury (RA; FIG. 1A) and right ventricular end diastolic pressure in millimeters of mercury (RVEDP; FIG. 1B), in accordance with various embodiments. A correction factor of 1.36 was used to convert the intracardiac pressures into centimeters of water in order to compare with JVP estimations.

FIGS. 2A and 2B illustrate the correlation between the NICHE™ algorithm and the hemodynamics RA (FIG. 2A) and RVEDP (FIG. 2B), in accordance with various embodiments.

FIGS. 3A and 3B illustrate the predictive strength of the NICHE™ algorithm (FIG. 3A) and the correlation between RVEDP and the NICHE™ algorithm (FIG. 3B), in accordance with various embodiments.

FIG. 4 illustrates the association between the NICHE™ groups and left ventricular filling pressures as measured by both the pulmonary artery diastolic pressure in millimeters of mercury (PADP) and pulmonary capillary wedge pressure in millimeters of mercury (PCWP), in accordance with various embodiments.

FIGS. 5A, 5B, and 5C illustrate representative ultrasound images of inspiration and expiration, in accordance with various embodiments. FIG. 5A illustrates representative ultrasound images from NICHE™ GROUP 1 (VC), FIG. 5B illustrates representative ultrasound images from NICHE™ GROUP 2 (VR), and FIG. 5C illustrates representative ultrasound images from NICHE™ GROUP 3 (NV). Abbreviations: IJ, internal jugular vein; VC, the IJ variation of respiration with collapse; VR, the IJ variation of respiration without collapse; NV, the IJ has no variations with respiration.

FIGS. 6A, 6B, and 6C illustrate the lack of reliability of NT-Pro BNP levels in assessing hemodynamics, in accordance with various embodiments. FIG. 6A illustrates N-terminal pro-brain-type natriuretic peptide (NT-proBNP) vs. RA, FIG. 6B illustrates NT-proBNP vs.RVEDP, and FIG. 6C illustrates BNP vs. PCWP; abbreviation: r, Spearman's rank correlation.

FIGS. 7a and 7B illustrate the correlation between the NICHE™ groups with both right- and left-sided filling pressures, in accordance with various embodiments. FIG. 7A illustrates RA vs. PWCP, and FIG. 7B illustrates RA and PWCP by NICHE™ group.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, methods, apparatuses, and systems are provided for non-invasive ultrasound assessment of intracardiac pressure via assessment of right internal jugular (IJ) vein characteristics. In exemplary embodiments, a computing device may be endowed with one or more components of the disclosed apparatuses and/or systems and may be employed to perform one or more methods as disclosed herein.

Embodiments herein provide methods, systems, and apparatus for non-invasive ultrasound assessment of intracardiac pressure. Embodiments provide assessment of right internal jugular (IJ) vein characteristics using a predictive model, the non-invasive cardiac hemodynamic evaluation (NICHE™) algorithm. These ultrasound assessments correlate with right heart catheterization (RHC)-measured intracardiac pressures.

According to various embodiments, the methods may be used for measuring a subject's right-sided intracardiac pressure and/or for assessing heart failure in a subject. The methods involve acquiring ultrasound image data of a right IJ vein in the subject, processing the ultrasound image data to determine vascular characteristic data for the IJ vein, and determining the right-sided intracardiac pressure from the vascular characteristic data. In some embodiments, the method also includes acquiring respiratory data corresponding to the IJ vein and determining the intracardiac pressure from both the vascular characteristic data and the respiratory data. In another embodiment, the method includes acquiring data indicative of velocity of blood flow in the IJ and determining the intracardiac pressure from both the vascular characteristic data and the blood flow velocity data.

According to some embodiments, the vascular characteristic data may include a maximum and a minimum cross-sectional area of the IJ vein lumen, and processing the ultrasound image data to determine vascular characteristic data may include calculating the difference between the maximum and minimum cross-sectional area of the IJ vein lumen over a time period, for instance a full expiration and inspiration cycle for the subject. Other embodiments include calculating the extent of collapse of the IJ vein over at least one full expiration and inspiration cycle of the subject, for example using the NICHE™ algorithm:

$$\frac{Surface {Area}_{inspiration} - Surface {Area}_{expiration}}{Surface {Area}_{inspiration}} \times 100$$

The techniques may be implemented according to various embodiments using an ultrasound device capable of imaging the right internal jugular vein or other vessels with which non-invasive pressure sensing is desired. The ultrasound device may be capable of imaging cross-sectional area variations of the IJ over an integration period (for instance, an inspiration-expiration cycle), which variations correspond to maximum and minimum cross-sectional areas in some embodiments. According to embodiments, the images may then be provided to an image processor or other computing device for determination of vascular characteristics based on the collected ultrasound image data.

Additionally, a respirometer may be used in certain embodiments to assess and record breathing conditions of the subject during the ultrasound imaging, for example, measuring the phase and volume of subject breathing, resulting in respiratory data. The respirometer may be a separate device in communication with the image processor device or may be combined with the ultrasound device. The respiratory data may then be provided to the predictive model executing on the image processor device.

The NICHE™ algorithm analyzes this respiratory data along with the vascular characteristic data from the ultrasound assessment. From this, the algorithm executed on the image processor may determine a predicted pressure in the IJ, which data may be displayed and reported to a physician on a display connected to the image processor. In other embodiments, the predictive model may separately determine a heart status assessment based on the calculated data. That assessment may be qualitative in nature and visually displayed to a physician, or that assessment may be used to automatically control diagnostic or surgical processes.

In an embodiment, data may be acquired that is indicative of the velocity of blood flow in the IJ. Such data may be incorporated in the NICHE™ algorithm to enhance the prediction of pressure in the IJ. The blood flow velocity data may be used in conjunction with the vascular characteristic data and, in an embodiment, with the respiratory data as well, to determine the intracardiac pressure.

Determination of blood flow velocity may be accomplished using Doppler, such as provided using an ultrasound device as described herein. In determining the velocity of the blood flow, either or both of a frequency shift or phase shift may be detected and quantified.

Clinical assessment of cardiac hemodynamics has always been challenging, and yet critical for the appropriate management of patients. Prior to this disclosure, the available methods for hemodynamic assessment were cumbersome, invasive, or unreliable. For instance, a commonly used physical examination metric for right-sided pressure assessment is the jugular venous pulsation (JVP). Traditionally, the assumption has been that the JVP estimation reliably predicts the central venous pressure (CVP), which in turn is considered to be relatively equivalent to right atrial pressure (RA), a marker of circulating volume and right ventricular function. However, the JVP estimation is limited by a number of factors such as the subject's body habitus, neck position, and operator interpretation and skill level—all factors that affect the reliability of this metric. Furthermore, clinical decision-making and management relying solely upon the estimation of the JVP may be inadequate or hazardous.

Currently, the gold standard and most direct method for measurement of intracardiac pressures is right heart catheterization (RHC). However, this procedure, by virtue of its direct pressure measurements in the heart, is invasive. As with most invasive procedures, RHC has its attendant risks and drawbacks, especially relevant in ill patient populations. And while RHC is generally safe, depending upon the clinical scenario, it is often cumbersome, time-consuming, and expensive. It is also impractical to perform serial RHC, and therefore the technique does not provide the immediate up-to-date clinical information necessary for management of patients on a day-to-day basis. Assessment of the right-sided intracardiac pressures is important in estimating right heart function and volume status in patients with chronic heart failure (CHF). It is also important in broad clinical settings, such as in volume management of chronic dialysis patients and critically-ill patients in the intensive care unit. Finally, given the invasive procedural nature of RHC, it is not an expedient, portable tool that lends itself to various clinical settings such as outpatient or clinic environments. Therefore, a reliable and portable non-invasive method of intracardiac hemodynamic assessment, such as those described herein, would have much clinical utility.

Thus, disclosed herein in various embodiments are methods of ultrasound (U/S) assessment of the internal jugular vein (IJ) that use a predictive algorithm that correlates with directly-measured intracardiac pressures via RHC. The algorithm, termed Non-Invasive Cardiac Hemodynamic Evaluation (NICHE™), predicts right-sided intracardiac pressures, reliably correlates with intracardiac hemodynamics, and is superior to alternative non-invasive methods and biomarkers. As described in various embodiments, the accuracy and validity of the NICHE™ algorithm was demonstrated by performing U/S on subjects who were simultaneously undergoing RHC.

In a specific, non-limiting embodiment, 42 consecutive subjects who presented to a cardiac catheterization laboratory for RHC were offered the NICHE™ method. Subjects with prior neck surgery, including carotid endarterectomy and thyroid surgery and those with indwelling neck catheters or known IJ thrombi were excluded. Informed consent was obtained according to a protocol approved by the Institutional Review Board at The University of Chicago Medical Center. Subject age, sex, height, weight, and medications were recorded. All measurements and clinical assessments were then made in the cardiac catheterization laboratory.

In this embodiment, to ensure standardization, the clinical assessment and estimation of the JVP was made with subjects lying on a 45° wedge-shaped pillow. Subjects were instructed to turn their head to the left to expose the right IJ. The height of pulsations of the jugular venous column was measured in centimeters from the angle of Louis and a standard 5 cm was added to this measurement and recorded as the JVP in cm H₂O. A correction factor of 1.36 (the conversion factor for the density of mercury to water) was used to convert the intracardiac pressures (in mmHg) in order to compare with JVP estimations (in cm H₂O). Hence, a RA pressure of 5 mmHg was deemed to be equivalent to 7 cm H₂O. Subjects were then positioned in a fully supine position without pillows.

Ultrasound evaluation of the right IJ was performed using a portable U/S device (Site˜Rite® V Ultrasound System, BARD Access Systems, Salt Lake City, Utah) with a vascular probe (solid-phase L-VA linear vascular probe for Site Rite 6 3-10 MHz). The probe was placed at the base of the sternocleidomastoid triangle with the subject's head turned to the left and images of the right IJ in cross-section were digitally captured. In a subgroup of subjects, resting and expiratory U/S images of the IJ were digitally captured and recorded for off-line analysis. The depth (cm) and diameter (cm) of the IJ were also recorded from the U/S images. Subjects were then asked to perform a deep inspiration with a short breath hold. Repeat U/S images of the IJ during inspiration were also captured for off-line analysis of respirophasic planimetry changes. Real time luminal characteristics of the IJ via U/S were then categorized and recorded into three groups as follows: GROUP 1 if the IJ expanded and collapsed with apposition by cross-section of the proximal and distal vessel walls, it was noted and recorded as variation of respiration with collapse (VC); GROUP 2 if the IJ lumen varied with respiration, but without collapse or apposition of the two walls and without lumen obliteration, then it was noted to have variations with respiration (VR); lastly, GROUP 3 if there were no significant visual changes in the luminal diameter of the IJ with respiration, then it was noted and recorded to have no variations with respiration (NV).

An a priori algorithm was generated, in this embodiment, using the real-time visual characteristics of the IJ in order to predict right-sided intracardiac pressures. The algorithm categorizes U/S characteristics of the IJ into three groups as described above and then predicts the RA pressure range based upon the group category. This algorithm is displayed in Table 1, below. NICHE™ GROUP 1 (or VC) was predicted to correlate with an RA pressure of 0-5 mmHg. NICHE™ GROUP 2 (or VR) was predicted to correlate with an RA pressure of >5 to ≦15 mmHg. NICHE™ GROUP 3 (or NV) was predicted to correlate with an RA pressure greater than 15 mmHg. All subjects were categorized in one of the three groups following U/S assessment of the IJ based upon this algorithm.

Immediately following U/S imaging and recording, with the subject remaining in a supine position, RHC was performed. Central venous access was obtained with a 7 French 10 cm IJ introducer sheath (Gordis Corporation, Miami Lakes, Fla.) via the right IJ using a modified Seldinger technique. Blood samples were obtained for laboratory testing if clinically indicated for the procedure. A sample of blood was obtained from a subset of 26 subjects for analysis of N-terminal pro-B-type natriuretic peptide (NT-ProBNP). A 7 Fr Swan Ganz catheter (Edwards Lifesciences LLC, Irvine, Calif.) was introduced through the venous sheath and advanced to pulmonary capillary wedge position with the use of hemodynamic and complimentary fluoroscopic guidance as needed. Non-invasive brachial artery blood pressures were measured, as were heart rate, rhythm, respirometry, and core temperature. The following intracardiac pressures were recorded: mean right atrial pressure (RA), right ventricular systolic pressure (RVSP) and end-diastolic pressure (RVEDP), pulmonary artery systolic and diastolic pressures (PAP), mean pulmonary artery pressure (MPA), and pulmonary capillary wedge pressure (PCWP). Cardiac output (CO) was determined in triplicate via thermodilution with the use of a GE Marquette Purka computer system, and pulmonary artery oxygen saturations were also obtained. At the time of measurement of right ventricular pressures, a small sample of blood was again taken from a subset of 14 subjects for analysis of N-terminal pro-B-type natriuretic peptide (NT-ProBNP). Blood pressure was recorded as systolic and diastolic (SBP and DBP) and mean arterial pressures (MAP).

The subject's most recent transthoracic echocardiogram (Phillips, Andover, Mass.) was reviewed in order to evaluate cardiac function and to determine the presence and extent of valvular abnormalities, including tricuspid regurgitation.

Static digital images of the IJ in short axis transverse-section were captured during the normal respiratory cycle in subjects and calibrated for circumference and area. The luminal surface areas of the IJ vessel in cross-section during inspiration and expiration were measured using SigmaScan Pro 5.0 software (Systat Software, Inc. San Jose, Calif.). The change in luminal surface area of the IJ vessel was calculated by the following formula to give a Collapsibility Index:

$$\frac{Surface {Area}_{inspiration} - Surface {Area}_{expiration}}{Surface {Area}_{inspiration}} \times 100$$

To compensate for variations in the size of IJ, the surface areas were each normalized to the vessel circumference of each subject. Mean change in luminal surface area was determined from the separate sections and used to calculate a group mean for each NICHE™ category.

Blood drawn from the venous sheath was sent for laboratory testing as part of standard clinical care. Testing included hemoglobin, creatinine and glomerular filtration rate calculations as per MDRD (Modification of Diet in Renal Disease). The blood drawn from the IJ from a random subset of 26 subjects was tested for N-terminal pro-B-type natriuretic peptide (NT-ProBNP) concentrations. Blood samples from the right ventricle were also collected and measured for NT-proBNP concentrations and compared with NT-proBNP concentrations from the IJ. Plasma NT-proBNP was determined using the two-site electrochemiluminescent assay on the Roche Elecsys platform (Basel, Switzerland).

Statistical analyses were performed using SigmaPlot (Systat Software, Inc. San Jose, Calif.) and STATA 10 (StataCorp, College Station, Tex.). Hemodynamic pressure measurements were expressed as mean values±SEM. Correlation of the NICHE™ algorithm and measured JVP and intracardiac hemodynamics were compared by ANOVA and Spearman's rank correlation. A p-value of ≦0.05 was considered statistically significant.

Table 1 represents the NICHE™ algorithm. Twenty-one subjects were in GROUP 1—respiratory variation with collapse on U/S assessment (VC), 17 subjects had respiratory variation without collapse (VR), and 4 subjects exhibited no respiratory variation in their IJ (NV). There were no significant differences in the clinical characteristics among these three groups, with similar BMI (p=0.79).

TABLE 1
NICHE ™ Algorithm: Prediction of Intracardiac Pressures

| | Right IJ Ultrasound | Predicted RA | # of |
|---|---|---|---|
| GROUP | Assessment at Inspiration | Pressure (mmHg) | Subjects |
| 1 | Respiratory variation with | 0-5 | 21 |
| | collapse (VC) | | |
| 2 | Respiratory variation without | >5-15 | 17 |
| | collapse (VR) | | |
| 3 | No respiratory variation (NV) | >15 | 4 |

Table 2 summarizes the baseline characteristics of all 42 subjects. The mean age of subjects was 53 years. Thirty-eight subjects (90%) were male, and the mean body mass index (BMI) was 27.5, which would be considered as overweight. All but two of the 42 subjects were cardiac transplant recipients. The transplant surgical techniques utilized were either total or bicaval anastomoses with routine concurrent DeVega tricuspid annuloplasty. There were no statistical differences in the clinical characteristics among the three NICHE™ groups, including renal function. There were also no statistical differences among the three groups of JVP.

TABLE 2
Clinical Characteristics

| | All | NICHE ™ | NICHE ™ | NICHE ™ | | JVP | |
|---|---|---|---|---|---|---|---|
| | Subjects | GROUP 1 | GROUP 2 | GROUP 3 | JVP <7 | 7-20 | JVP>20 |
| | (n = 42) | (21) | (17) | (4) | (13) | (24) | (5) |
| Age | 53.1 ± 12.9 | 53.57 ± 13.0 | 55.70 ± 10.89 | 39.25 ± 14.93 | 58 ± 7.55 | 51.96 ± 14.27 | 45.6 ± 14.57 |
| Weight | 83.85 ± 21.24 | 85.62 ± 16.6 | 83.62 ± 14.78 | 95.22 ± 24.99 | 77.03 ± 12.40 | 92.07 ± 53.64 | 77.91 ± 11.74 |
| (kg) | | | | | | | |
| Height | 1.72 ± 0.28 | 1.76 ± 0.08 | 1.77 ± 0.11 | 1.734 ± 0.14 | 1.71 ± 0.08 | 1.80 ± 0.90 | 1.73 ± 0.12 |
| (m) | | | | | | | |
| BMI | 27.5 ± 4.5 | 27.4 ± 4.4 | 26.6 ± 4.2 | 31.2 ± 5.7 | 26.1 ± 5.5 | 28.4 ± 5.1 | 25.7 ± 1.8 |
| Cr | 1.6 ± 1.0 | 1.4 ± 0.6 | 1.9 ± 1.5 | 1.5 ± 0.3 | 1.4 ± 0.6 | 1.7 ± 1.2 | 1.6 ± 0.6 |
| (mg/dL) | | | | | | | |
| GFR | 55.57 ± 22.46 | 61.0 ± 20.70 | 70.70 ± 25.72 | 47.75 ± 9.42 | 60.31 ± 22.30 | 54.29 ± 23.13 | 49.4 ± 21.73 |
| (ml/min) | | | | | | | |

Abbreviations:
BMI, body mass index, calculated as weight in kilograms divided by height in meters squared;
Cr, creatinine in milligrams per deciliter;
GFR, glomerular filtration rate in millimeters per minute.

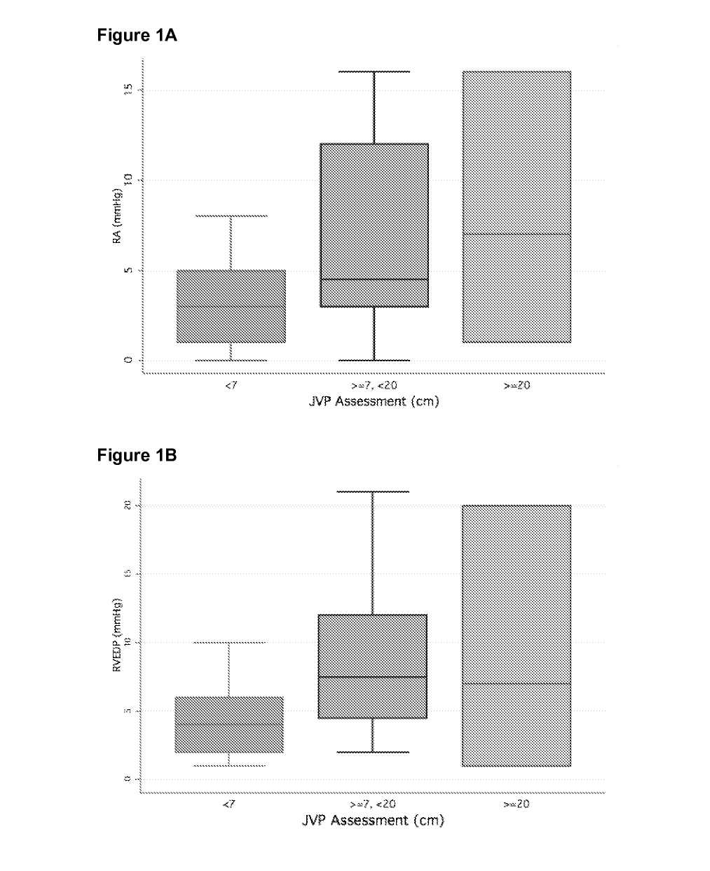

To determine the accuracy of JVP estimation, JVP was compared in all subjects with catheter-based hemodynamic pressures. The correlation between the standard method of JVP estimation and intracardiac hemodynamics can been seen in Table 3 and FIG. 1. In order to compare JVP estimations with the three NICHE™ category groups of RA pressure (≦5, >5 to ≦15, >15 mmHg), a conversion factor of 1.36 was used as described above in order to standardize units. The adjusted NICHE™ RA pressure groups are as follows: <7, ≧7 to <20 and >20 cm H₂O. The JVP was estimated to be less than 7 cm H₂O in 27 subjects. In 12 subjects, the JVP was estimated to be ≧7 and <20 cm H₂O, and in 3 subjects the JVP was estimated to be greater than 20 cm H₂O. There was no difference in median BMI between these groups (p=0.42). As shown in Table 3 and FIG. 1, JVP correlated poorly with invasive intracardiac hemodynamics. The mean difference between the estimated JVP and actual measured RA pressure was 5.3±4.9 cm H₂O. Only 14 of 27 (52%) JVP estimations accurately predicted that the RA was <7 cm H₂O, while only 7 of 12 (58%) JVP estimations accurately predicted the RA to be ≧7 and <20 cm H₂O, and finally only ⅓ in the JVP estimations correctly identified an RA of >20 cm H₂O. Similarly, the JVP estimations did not correlate well with RVEDP (FIG. 1).

TABLE 3
JVP vs. Intracardiac Pressures

| | RA (mmHg) | | | RVEDP (mmHg) | | |
|---|---|---|---|---|---|---|
| | ≦5 | >5-15 | >15 | ≦5 | 5-15 | >15 |
| JVP <7 | 14 | 12 | 1 | 16 | 10 | 1 |
| JVP 7-20 | 3 | 7 | 2 | 6 | 4 | 2 |
| JVP >20 | 1 | 1 | 1 | 1 | 1 | 1 |

A correction factor of 1.36 was used to convert the intracardiac pressures into centimeters of water in order to compare with JVP estimations.

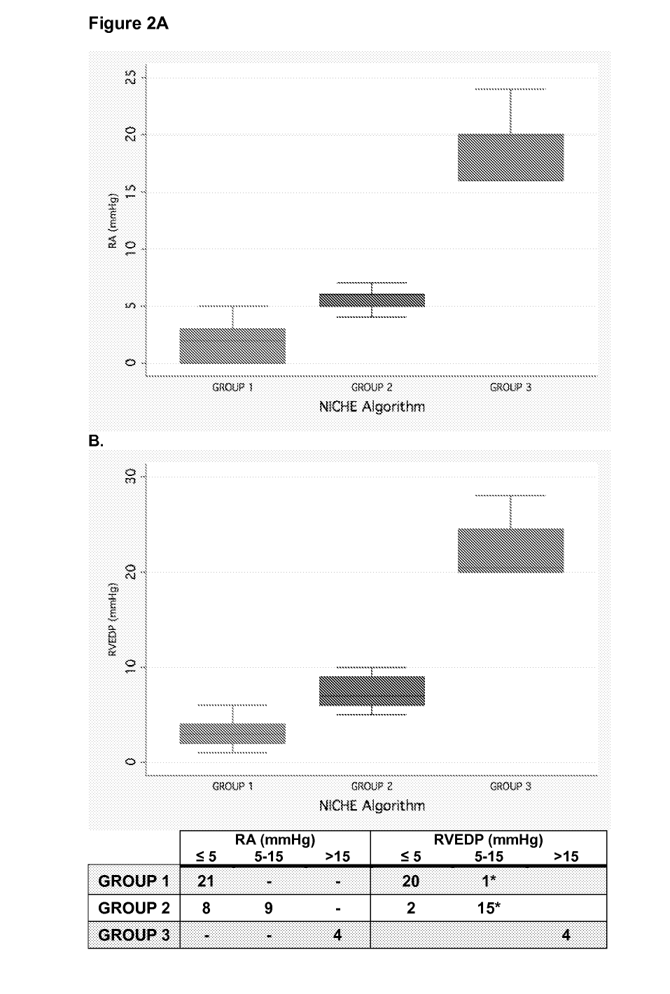

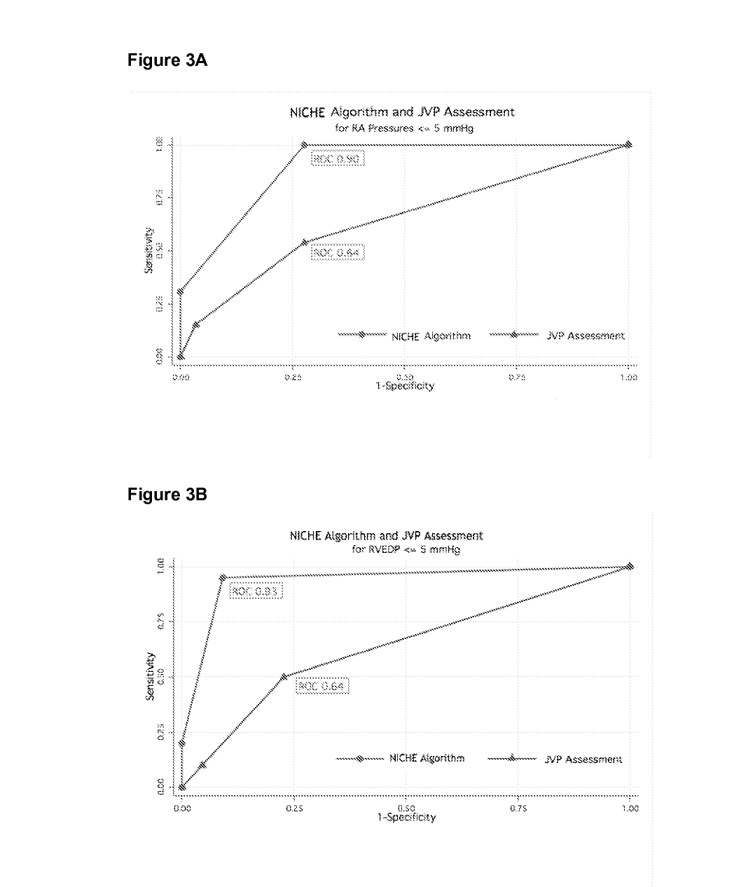

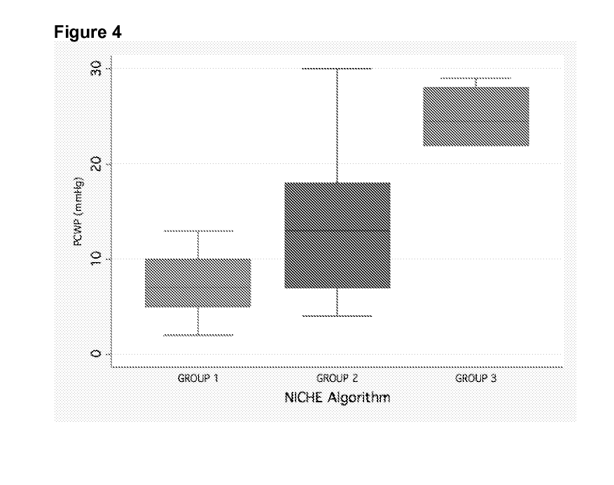

To demonstrate that the NICHE™ algorithm accurately predicts right-sided hemodynamics, the NICHE™ groups were compared with catheter-based hemodynamics. Unlike JVP estimations, there was strong correlation between the NICHE™ algorithm and hemodynamics, especially for RA and RVEDP (Table 4 and FIG. 2). In fact, every subject placed in NICHE™ GROUP 1 (respiratory variation with collapse) had an RA pressure of ≦5 mmHg. The predictive strength of the NICHE™ algorithm for the NICHE™ GROUP 1 (an RA<5 mmHg) was 90% sensitive, and 91% specific (PPV of 83%, NPV of 89%) as shown in FIG. 3A. The strength of the association was even greater for RVEDP. Of the 21 subjects placed into NICHE™ GROUP 1, 20 had an RVEDP of ≦5 mmHg. In 15 of 17 subjects, the NICHE™ GROUP 2 accurately predicted the RVEDP to be >5 and ≦15 mmHg and all 4 subjects placed into NICHE™ GROUP 3 had an RVEDP>15 mmHg. The correlation between RVEDP and the NICHE™ algorithm showed a sensitivity of 86% and a specificity of 100% for NICHE™ GROUP 1 (PPV of 100% and NPV of 88%) as shown in FIG. 3B. The RV systolic pressure and pulmonary artery systolic pressure were not statistically different between the three groups, although they were higher in those subjects with higher diastolic filling pressures (Table 4). Surprisingly, the NICHE™ groups also showed an association with left ventricular filling pressures as measured by both the PADP and PCWP (FIG. 4).

Right ventricular (RV) function has prognostic significance in heart failure, and RV dysfunction has been shown to predict reduced exercise capacity and survival. Non-invasive methods to evaluate RV function include echocardiography magnetic resonance imaging, high frequency thermodilution, contrast ventriculography, and radionucliotide ventriculography. However, none of these techniques have proven to be a reliable and validated method for right ventricular function. Standard transthoracic echocardigraphy is currently the most common imaging modality to assess RV function.

As described herein, there was a strong correlation of the NICHE™ algorithm with RVEDP. During ventricular diastole, the IJ volume should reflect the pressure and volume of the right ventricle. In animal models, the RA pressure returns to baseline more quickly than both the inferior and superior vena cava (SVC) following both volume and vasopressor challenge. Similarly, studies of RA pressures in microgravity showed an increase in atrial pressures despite lower CVP (or SVC) pressures during weightlessness. Without being bound by theory, the fact that the correlation of the NICHE™ algorithm with the RVEDP was even more robust than the correlation with the RA may be indicative of the anatomical nature of the RA with its network of pectinate muscles. The pectinate muscles are easily distensible and compliant in their design to provide even and constant venous return to the right ventricle and left heart.

The hemodynamic data indicates that the RV functions along a Starling curve and when there is insufficient preload of the right ventricle, the cardiac output is less than when the RV is adequately filled. The well-described algorithm for early goal directed therapy in sepsis used CVP as one of the branch points for intervention, with <8 mmHg as the clinical criteria for aggressive volume replacement. In certain embodiments, the NICHE™ algorithm may be used to determine this branchpoint non-invasively.

TABLE 4
Intracardiac Pressures by NICHE ™ Algorithm

| | GROUP 1 | GROUP 2 | GROUP 3 | p Value |
|---|---|---|---|---|
| | (21) | (17) | (4) | for difference |
| RA, mmHg | 1.95 ± 1.71 | 5.88 ± 2.83 | 18 ± 4 | 0.04 |
| RVSP, mmHg | 27.10 ± 6.22 | 34.18 ± 8.16 | 54.75 ± 4.5 | 0.35 |
| RVEDP, mmHg | 2.95 ± 1.50 | 7.70 ± 2.31 | 22.25 ± 3.86 | 0.03 |
| PASP, mmHg | 24.48 ± 7.68 | 33.17 ± 9.34 | 53.5 ± 5.91 | 0.54 |
| PADP, mmHg | 9.29 ± 4.38 | 14.29 ± 6.83 | 25.25 ± 1.89 | 0.03 |
| PA Mean, mmHg | 16.14 ± 5.59 | 22.53 ± 7.53 | 38 ± 2.16 | 0.08 |
| PCWP, mmHg | 7.48 ± 3.21 | 13.53 ± 7.72 | 25 ± 3.56 | 0.018 |
| MAP, mmHg | 104.35 ± 13.27 | 93.23 ± 26.40 | 96.25 ± 14.34 | 0.02 |
| CO, L/min | 5.87 ± 0.94 | 6.27 ± 1.39 | 5.22 ± 2.78 | 0.04 |
| CI, L/min/m² | 2.92 ± 0.46 | 3.138 ± 0.75 | 2.54 ± 1.13 | 0.03 |
| SVR, | 1434 ± 334 | 1206 ± 225 | 1584 ± 1146 | 0.14 |
| dynes/sec/cm⁻⁵ | | | | |
| PVR, WU | 1.5 ± 0.8 | 1.5 ± 0.6 | 2.9 ± 1.5 | 0.71 |

Abbreviations:
RVSP, right ventricular systolic pressure in millimeters of mercury;
RVEDP, right ventricular end diastolic pressure in millimeters of mercury;
PASP, pulmonary artery systolic pressure in millimeters of mercury;
PADP, pulmonary artery diastolic pressure in millimeters of mercury;
PA, pulmonary artery pressure in millimeters of mercury;
PCWP, pulmonary capillary wedge pressure in millimeters of mercury;
MAP, mean arterial pressure in millimeters of mercury;
CO, cardiac output in liters per minute;
CI, cardiac index in liters per minute per meter squared;
SVR, systemic vascular resistance in dynes per second per centimeter to the minus fifth;
PVR, pulmonary vascular resistance in Woods units.

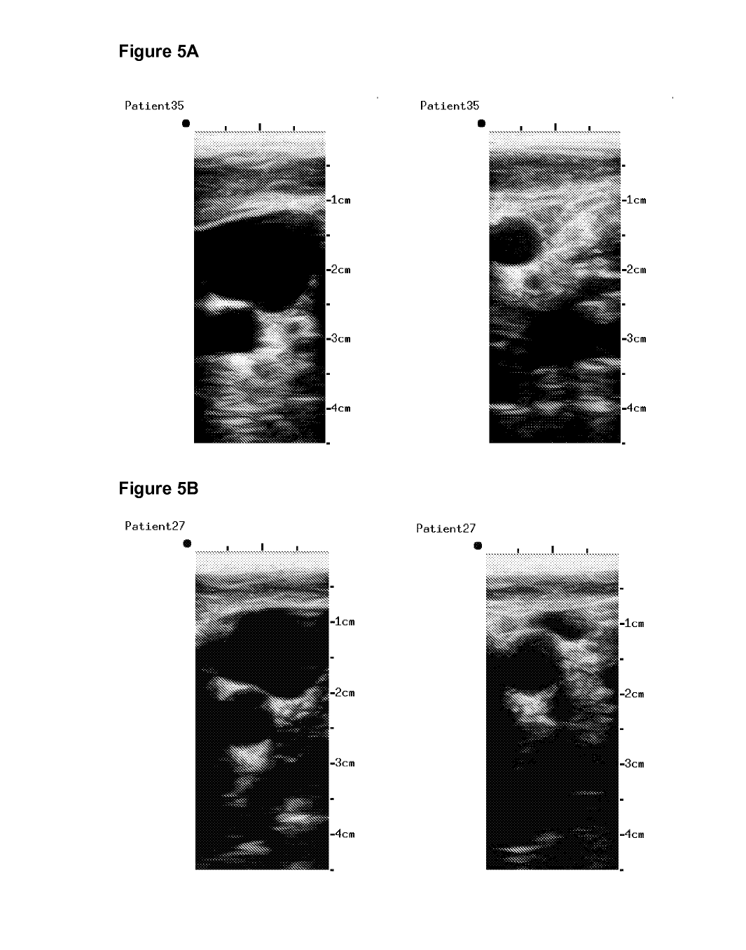

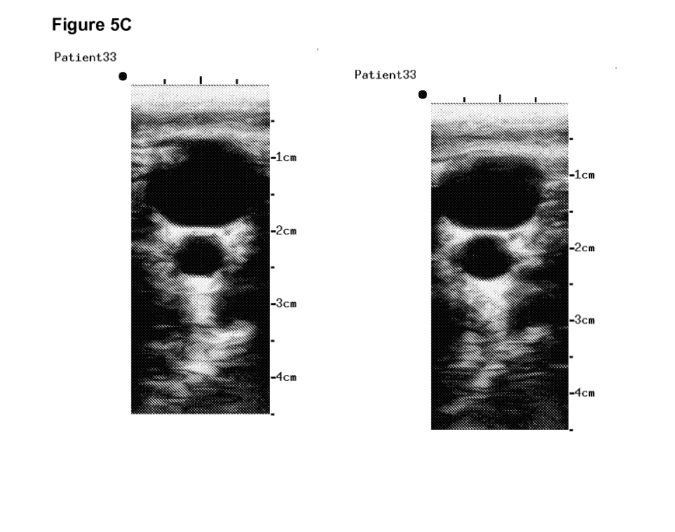

The categorization of IJ vessels into the NICHE™ groups via U/S was performed visually by several operators as described above. In order to determine whether exact quantification of the NICHE™ algorithm group assignments was possible and equivalent, digital planimetry was performed on IJ vessels. Digital images were captured in 26 subjects and then recorded for off-line analysis. In 23 subjects, there was an adequate set of both inspiratory and expiratory images. Representative U/S images of inspiration and expiration are shown in FIG. 5. In 23 expiratory images of the IJ, the mean surface area was 1.13±0.63 cm². The mean surface area of the 23 inspiratory images was 0.42±0.52 cm². The total mean percent change in surface area of all sets of images was 63.3±29.83%. The mean percent change in surface area for NICHE™ GROUP 1 was 64.48±32.07% while the mean percent change in surface area for NICHE™ GROUP 2 was 65.29±27.58%. Only one subject in NICHE™ GROUP 3 had adequate images and the mean percent change was 30.32%.

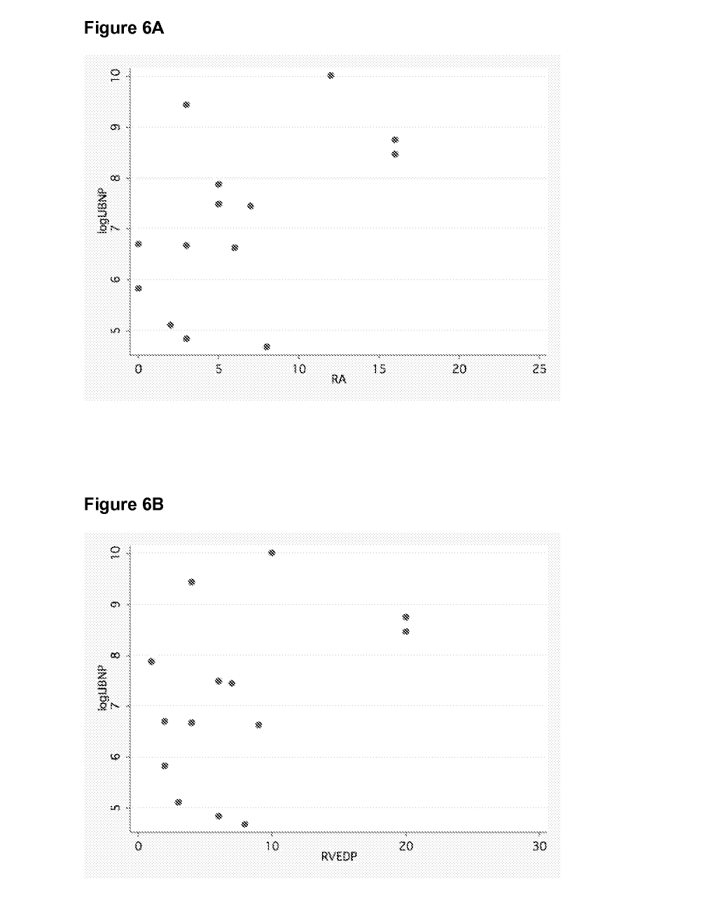

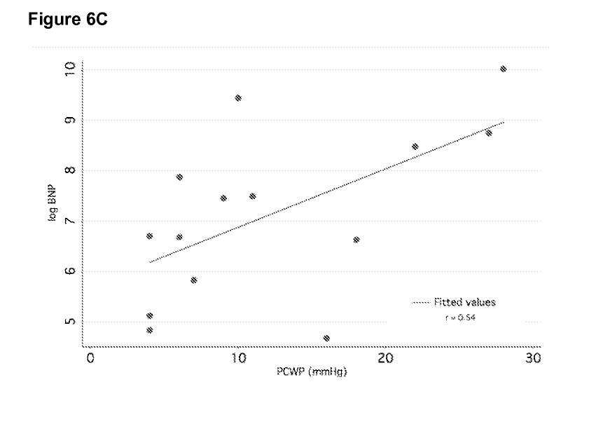

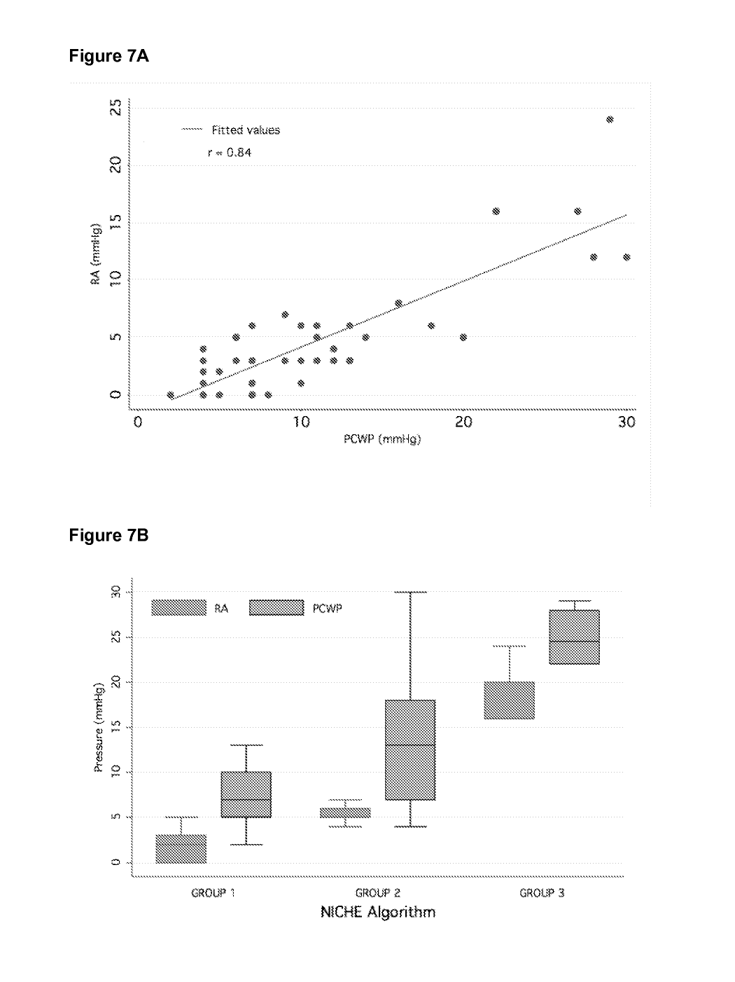

To determine if NT-proBNP, a serum biomarker of hemodynamics, accurately correlated with intracardiac pressures, NT-proBNP measurements were drawn and analyzed. There was no significant difference between the NT-proBNP measured from the IJ with the NT-proBNP measured from the RV. In comparing the NT-Pro BNP levels with the catheter-measured RA pressure and the NICHE™ algorithm, it was clear that there were no obvious correlations, r=0.29 and r=0.14 respectively (FIG. 6). These results confirm that NT-Pro BNP levels are unreliable in assessing hemodynamics, especially in certain populations.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method for assessing right-sided intracardiac pressure in a subject, comprising:
acquiring ultrasound image data of a right internal jugular (IJ) vein in the subject;
processing the ultrasound image data to determine vascular characteristic data for the IJ vein; and
determining the right-sided intracardiac pressure from the vascular characteristic data.

2. The method of claim 1, further comprising:
acquiring respiratory data corresponding to the IJ vein; and
determining the intracardiac pressure from both the vascular characteristic data and the respiratory data.

3. The method of claim 1, further comprising:
acquiring data indicative of blood flow velocity in the IJ vein; and
determining the intracardiac pressure from both the vascular characteristic data and the blood flow velocity data.

4. The method of claim 1, further comprising displaying the intracardiac pressure on an ultrasound monitor.

5. The method of claim 1, further comprising recording the intracardiac pressure.

6. The method of claim 1, wherein the vascular characteristic data comprises a maximum and a minimum cross-sectional area of the IJ vein lumen.

7. The method of claim 6, wherein processing the ultrasound image data to determine vascular characteristic data comprises calculating the difference between the maximum and minimum cross-sectional area of the IJ vein lumen over a time period.

8. The method of claim 7, wherein the time period spans at least one full expiration and inspiration cycle for the subject.

9. The method of claim 8, wherein processing ultrasound image data further comprises calculating the extent of collapse of the IJ vein over the at least one full expiration and inspiration cycle of the subject.

10. The method of claim 9, wherein calculating the extent of collapse of the IJ vein comprises calculating a difference between an inspiration and expiration surface area divided by the inspiration surface area.

11. A method for assessing heart function in a subject, the method comprising:
acquiring ultrasound image data of the subject's vasculature;
processing the ultrasound image data to identify IJ vessel size data over an integration period; and
determining an indicator of heart status from the IJ vessel size data.

12. The method of claim 11, wherein the indicator of the heart status is a measure of IJ vessel collapse.

13. The method of claim 11, wherein the integration period comprises at least one full inspiration-expiration cycle of the subject.

14. The method of claim 11, wherein the indicator of the heart status is an estimate of right-sided intracardiac pressure.

15. The method of claim 11, wherein the indicator of the heart status is a measure of heart failure.

16. A system for executing the method of claim 1.

17. The system of claim 16 comprising:
an ultrasound imaging apparatus for capturing the ultrasound image data;
an image processor for receiving and analyzing the ultrasound image data; and
a display.

18. An apparatus comprising:
a computer-readable medium including instructions, which, when executed by a computing device, enable the computing device to perform operations comprising:
acquiring ultrasound image data of an IJ vein;
processing the ultrasound image data to determine vascular characteristic data for the IJ vein; and
determining right-sided intracardiac pressure from the vascular characteristic data.

19. The apparatus of claim 18, wherein determining right-sided intracardiac pressure comprises calculating the extent of collapse of the IJ vein over at least one full expiration and inspiration cycle.
